(19) United States
(12) Patent Application Publication
(10) Pub. No.: US 2007/0064867 A1
(43) Pub. Date: Mar. 22, 2007

(54) Apparatus and method to acquire data for reconstruction of images pertaining to functional and anatomical structure of the breast

(76) Inventors: Timothy B. Hansen, Fort Lauderdale, FL (US); Heang K. Tuy, Sunrise, FL (US); Robert H. Wake, Cooper City, FL (US); Steven L. Ponder, Ft. Lauderdale, FL (US); Patrick Olivier, Plantation, FL (US)

Correspondence Address: SHLESINGER, ARKWRIGHT & GARVEY LLP;Suite 600, 1420 King Street, Alexandria, VA, 22314, US

(21) Appl. No.: 11/522,923

(22) Filed: Sep. 19, 2006

Related U.S. Application Data
(60) Provisional application No. 60/718,307, filed on Sep. 20, 2005.

(51) Int. Cl. A61B 6/04

(52) U.S. Cl. 378/37

(57) ABSTRACT

An apparatus for breast scanning to obtain functional and anatomical images of the breast comprises a patient support for a patient to rest in a prone position, the support having an opening with one of her breasts vertically pendent through the opening for scanning; a laser CT scanner disposed below the support for generating a first set of data for reconstruction of functional images of the breast; an X-ray CT scanner disposed below the support for generating a second set of data for reconstruction of anatomical images of the breast; and a display to visualize at least one of the functional and anatomical images. A method for acquiring data for reconstruction of images pertaining to functional and anatomical structures of a breast comprises positioning a patient in a prone position on a support having an opening through which a breast of the patient is pendant; scanning the breast with a laser CT scanner to obtain data of the breast for functional image reconstruction of the breast; and while the patient is still prone on the support, scanning the breast with an X-ray CT scanner to obtain data of the breast for anatomical image reconstruction of the breast.

### RELATED APPLICATIONS

This is a nonprovisional application, which claims the priority benefit of provisional application Ser. No. 60/718,307, filed Sep. 20, 2005, herein incorporated by reference.

### BACKGROUND OF THE INVENTION

It is vital to be able to detect cancers in the breast at their early stage. Moreover, it is very important to discern malignant from benign lesions. For biopsy and surgery purposes, it is essential to know the location and the extent of the lesion. For therapeutic purposes, it is useful to track tumor response to neoadjuvant and radiation therapy.

Current commercial scanners or medical diagnostic equipments available in the market provide only partial solutions to the above goals. For instance, it is current practice to use mammography to detect tumors. A major drawback of this imaging modality is that each pixel of a mammography image represents a ray sum of attenuation coefficients of the tissue along the ray. Therefore, the 3D location of the tumor or the depth effect is lost. The compression employed in mammography causes pain and disturbs the breast, making it difficult to determine biopsy sites and has the risk of potentiating the metastatic spread of cancer. This deficiency may be overcome by using ordinary CT scanners. Unfortunately, a much higher x-ray dose to the patient compared to that of mammography is inherent in the conventional CT scans. To address the issues on overdosing the patient, J. Boone et al. (Radiology, 221, 657-667, 2001), have conducted extensive studies on using the x-ray cone beam CT technology with a special positioning of the patient in order to expose x-ray to the breast area only.

At this point, it is worth noting that both the x-ray mammography and CT modalities may be able to detect a lesion inside the breast, but neither of them is able to distinguish benign from malignant lesions. The distinction between these lesions could be based on the angiogenesis. Current development of CT scanners by Imaging Diagnostic Systems, Inc. (U.S. Pat. Nos. 5,692,511; 6,130,958 and 6,211,512), using a laser energy source instead of x-ray have shown the capability of providing a map of the blood supply (hemoglobin), which is present to feed the lesion. This extremely useful information suffers from the limited spatial resolution caused by the scatter effect of the laser while penetrating inside the breast, and the aberration effect due to the non linear photon migration path. To improve the limited resolution of such imaging system and to speed up the computational reconstruction time, Kawaguchi et al. (U.S. Pat. No. 5,419,320) suggested generating functional images based on an x-ray CT scan of the same anatomy. This method relies on an initial estimate of the functional images using the standard values of physical quantities of the corresponding anatomical structures identified by an image segmentation of the x-ray CT images. The final functional images are computed iteratively to match the optical data collected during the laser scan. One disadvantage of this method is that it would not be possible to generate functional images without the availability of the corresponding anatomical images from the x-ray scan. Moreover, an estimate of a functional image from an anatomical image is not reliable. This could affect the convergence of the algorithm, and may lead to a false functional image.

### OBJECTS AND SUMMARY OF THE INVENTION

It is an object of the present invention to provide an apparatus for providing functional and anatomical images of the breast for cancer screening and diagnosis.

It is another object of the present invention to provide an apparatus for generating images of the anatomical structure of the breast with minimal dose to the patient to be an effective screening tool for breast cancer detection.

It is still another object of the present invention to provide an apparatus for providing functional and anatomical images of the breast that are correlated to provide complementary information for screening and diagnosis of breast cancer.

It is an object of the present invention to provide an apparatus for providing functional and anatomical images of the breast that provide independent scanning with laser and X-ray to complete a scan relatively quickly to minimize patient motion.

It is another object of the present invention to provide an apparatus for providing functional and anatomical images of the breast using laser and X-ray scanners with independent image reconstruction.

In summary, the present invention provides an apparatus for breast scanning to obtain functional and anatomical images of the breast, comprising a patient support for a patient to rest in a prone position, the support having an opening with one of her breasts vertically pendent through the opening for scanning; a laser CT scanner disposed below the support for generating a first set of data for reconstruction of functional images of the breast; an X-ray CT scanner disposed below the support for generating a second set of data for reconstruction of anatomical images of the breast; and a display to visualize at least one of the functional and anatomical images.

The present invention also provides a method for acquiring data for reconstruction of images pertaining to functional and anatomical structures of a breast, comprising positioning a patient in a prone position on a support having an opening through which a breast of the patient is pendant; scanning the breast with a laser CT scanner to obtain data of the breast for functional image reconstruction of the breast; and while the patient is still prone on the support, scanning the breast with an X-ray CT scanner to obtain data of the breast for anatomical image reconstruction of the breast.

These and other objects of the present invention will become apparent from the following detailed description.

### BRIEF DESCRIPTION OF THE DRAWINGS

FIG. 1 is a block diagram of a scanning apparatus made in accordance with the present invention.

FIG. 2 is a schematic perspective view of the scanning apparatus, showing both the laser and X-ray CT scanners made in accordance with the present invention.

FIG. 3 is a schematic perspective view of the main components of an X-ray CT scanner shown in FIG. 2.

FIG. 4 is a top view of FIG. 3, showing the breast within the X-ray scanner scanning field.

FIG. 5 is a schematic perspective view of the main components of a laser CT scanner shown in FIG. 2.

FIG. 6 is a top view of FIG. 5, showing the breast within the ring of detectors of the laser CT scanner.

FIG. 7 is a top view of both the laser and x-ray CT scanners in relation to the breast.

FIG. 8 is a block diagram of a process representing the main components in the image reconstruction from data collected by the x-ray CT scanner.

FIG. 9 is a block diagram of a process representing the main components in the image reconstruction from data collected by the laser CT scanner.

FIG. 10 is a continuous wave spectrophotometry of various materials.

### DETAILED DESCRIPTION OF THE INVENTION

A scanning apparatus made in accordance with the present invention comprises two independent CT scanners sharing a patient couch. The patient lies on the couch in a prone position, with one of the breasts vertically pendent through an opening in the couch for scanning. The two scanners share a common patient couch to facilitate a direct correlation of the reconstructed images representing the functional and anatomical structures of the breast that are derived from the data collected by the two scanners respectively. The laser scan of the breast is followed immediately by an x-ray scan or vice-versa in order to keep the patient position invariant. To advantageously shorten the total scan time, the two scans may be performed concurrently. Two independent image reconstruction systems are provided. The first is for the reconstruction of functional images from data collected by the laser CT scanner, and the other for the reconstruction of anatomical images from data collected by the x-ray CT scanner. To allow a physician to detect, locate and discern cancer cells inside the breast, the reconstructed images are displayed in 2D and 3D format. The images may be displayed separately or concurrently using image fusion based on the physical location of the cross-sections of the breast along which both sets of images were reconstructed. The fused images enable the visualization of the sets of images separately or concurrently in order to facilitate detection of cancer, its location and extent inside the breast.

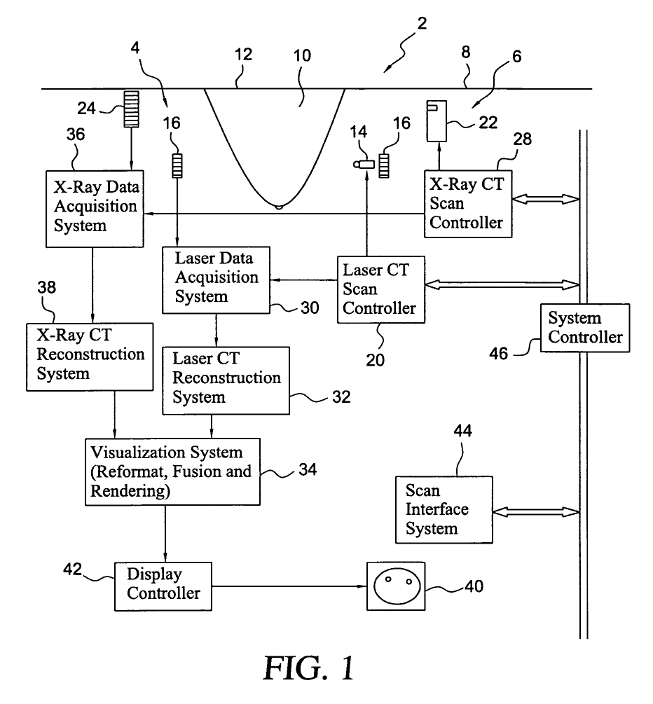

Referring to FIG. 1, an apparatus 2 made in accordance with the present invention is disclosed. The apparatus 2 includes a laser CT scanner 4 and an X-ray scanner 6. The laser and X-ray CT scanners 4 and 6 are disposed below a patient couch 8. The patient lies on the couch 8 in a prone position, having her breast 10 vertically pendent through an opening 12 in the couch 8. The couch 8 is shielded according to standard methods to ensure that only the scanned breast is exposed to the x-ray radiation during the scan in order to minimize the radiation dose to the patient.

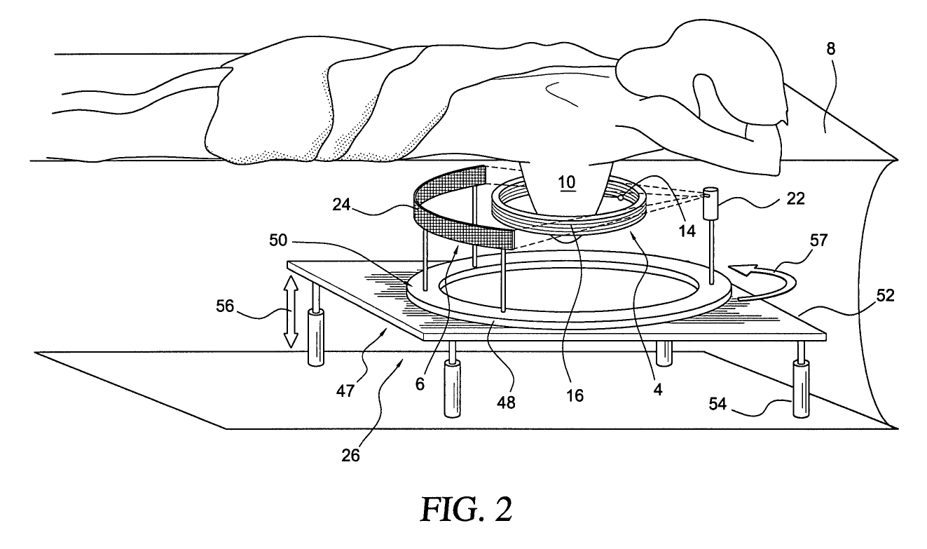

The laser CT scanner 4 includes a laser source 14 and a ring of multiple rows of detectors 16 (also see FIG. 2). The laser source 14 is mounted to a fixture (not shown), as will be explained below, that allows a helical movement of the laser source 14 during a scan. The ring of detectors 16 is mounted to the same fixture (not shown), as will be explained below, that allows a linear, up and down motion, during the scan. The movements of the laser source 14 and the detector ring 16 are synchronized in such a way that the laser source remains in a middle plane defined by a middle row of detectors in the detector ring. These movements are monitored by a laser scan controller 20.

The laser CT scanner 4 provides the collection of data for reconstruction of functional images of the breast. In medical imaging, functional images show the body at work. Examples of functional images are those showing blood flow, brain activities, oxygen consumption, oxy-hemoglobin increase, or what a tumor is doing to a body. In the case of laser tomography for breast cancer detection, an objective is to image pools of blood feeding cancer cells in the breast. This is done using the fact that blood absorbs more photons from the laser source than regular breast tissue does, causing less photons received at the detectors surrounding the breast.

The x-ray CT scanner 6 includes an x-ray source 22 and an arc of multiple rows of detectors 24. Both the x-ray source 22 and the detector arc 24 are mounted to a fixture 26 (see FIG. 2), allowing a helical movement during a scan of the breast. This movement is monitored by an X-ray scan controller 28.

Functional data are collected by a laser data acquisition system 30 during an optical scan when the laser source 14 emits a pencil beam continuously toward the breast. To determine the boundary of the breast, a portion of the light reflected from the incidence of the laser beam with the breast is recorded by two CCD cameras mounted near the laser source, as described in U.S. Pat. No. 6,044,288. For the sake of clarity, the cameras are not shown. This data set is also collected by the laser data acquisition system 30. These two sets of data are then fed to a laser CT reconstruction system 32, which is responsible for generating functional images along a plurality of cross sections of the breast, as described in U.S. Pat. No. 6,130,958. The reconstructed functional images are in a format readily available to be displayed by a visualization system 34. The laser CT scan controller 20 supervises the laser data acquisition system 30.

Anatomical data are collected by an X-ray data acquisition system 36 during an X-ray CT scan when the X-ray source 22 emits a limited cone beam toward the breast. This data set is fed to an x-ray CT reconstruction system 38, which is responsible for generating anatomical images along a plurality of cross sections of the breast. The reconstructed anatomical images are in a format readily available to be displayed by the visualization system 34. The x-ray CT scan controller 28 supervises the x-ray data acquisition system 36.

The visualization system 34 is responsible for displaying functional and anatomical images in various formats, including cross-section, sagittal, coronal, or 3D views. The 3D views may be in the form of surface shading, maximum intensity projection (MIP) or volume rendering (VR). The functional anatomical images may be displayed separately or concurrently using an image fusion process based on the exact physical location of the cross-sections of the breast along which both sets of images were reconstructed.

The X-ray CT scanner 6 produces images showing anatomical structure of the breast including, for example, fat, soft tissue, blood vessels, etc. If there are tumors, the tumors are shown in the CT images. However, both benign and malignant tumors are shown the same way so that it is very difficult, if not impossible, to distinguish them by viewing CT images. Functionally, malignant tumors require blood for their growth. The blood concentration feeding the tumors can be picked up by the laser CT scanner 4 and shown in the laser CT images, but not the tumors themselves. A way to correlate the tumors with their blood supply is through their relative locations within the images from the two modalities.

X-ray CT images (anatomical images) and laser CT images (functional images) of the breast constitute two sets of slices reconstructed from the data collected by the corresponding scanners. In both cases, the slice locations are known from the reconstruction process and the data collection. From this knowledge, the slices from these 2 sets of images are correlated. Image slice interpolations within one set of images may be required if the images of the 2 sets were not reconstructed at the same slice locations.

Consequently, it is advantageous to combine two images from the 2 sets at the same slice location, creating another image showing the characteristics of both original images at the same time. A linear combination of the grey level (image intensity) of the two images at the same pixel location is commonly used in this process. The resulting image will show the tumors from the x-ray CT image and the blood concentration from the laser CT image within the same area if the tumors are malignant.

Final images prepared by the visualization 34 are displayed on a single or multiple display monitors 40 via a display controller 42.

An operator controls or selects a mode of operation of the apparatus 2 via a scan user interface system 44, which extracts relevant parameters from the user input and passes them on to a system controller 46. Using these parameters, the system controller 46 controls and monitors the operations of the scanner by issuing appropriate commands to either the laser CT controller 20 or X-ray CT controller 28. The status of the scanner is fed back from both the laser and x-ray CT controllers to the system controller. Some of the status may be fed back to the operator via the scan user interface system 44.

Referring to FIG. 2, a schematic perspective view of the apparatus 2 is disclosed, showing a patient lying in a prone position on the couch 8. Underneath the couch are the laser and X-ray CT scanners 4 and 6.

The X-ray source 22 and the arc of detectors 24 are attached to a mechanical structure 47 comprising a rotor 48 of a bearing 50. The arc of detectors 24 and the X-ray source 22 are attached to the rotor 48, enabling a circular motion for scanning. A stator 52 of the bearing is supported by four vertical actuators 54, which facilitate a linear, up and down motion 56 during the scan. The rotation 57 of the rotor 48 and the linear, up and down motion 56 of the stator 52 provide a helical movement of the x-ray source 22 and the detectors 24 for scanning purpose.

A mechanical structure similar to the structure 47 comprising a bearing and linear up and down actuators, but smaller in size is provided to support the laser source 14 and detector ring 16, enabling a helical motion of the laser source and the detector ring 16 for scanning. For the sake of clarity, this mechanical structure is not shown in the figure.

The helical movements supported by the two above mechanical fixtures are preferably decoupled in order to provide independent scans by the laser and X-ray CT scanners.

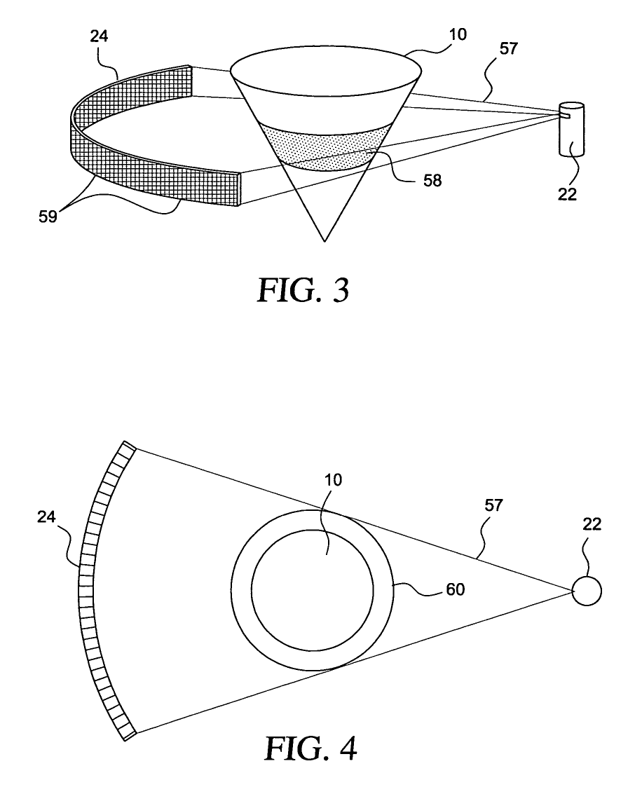

FIG. 3 shows the extent of a limited cone beam 57 of X-ray radiation required for scanning. The limited cone beam 57 is realized by placing a collimator, not shown in the figure, located near the X-ray tube 22, right in front of the X-ray focal spot. At one particular instance of time during a scan, an area 58 of the breast 10, is exposed to the radiation dose, depending on the location of the x-ray tube 22 and that of the arc of detectors 24. The arc of detectors comprises multiple rows of individual detectors 59. The limited cone beam 57 is further disclosed in co-pending application Ser. No. 11/494,534, filed Jul. 29, 2006, herein incorporated by reference. As discussed in the cited co-pending application, a limited cone beam is advantageously used to reduce the radiation dose to the patient.

FIG. 4 is a schematic top view of FIG. 3, showing the extent of the limited cone beam 57. The x-ray tube 22 and the detector area 24 are positioned so that any cross-section of the breast 10 would be within a region 60 within the boundary of the cone beam 57.

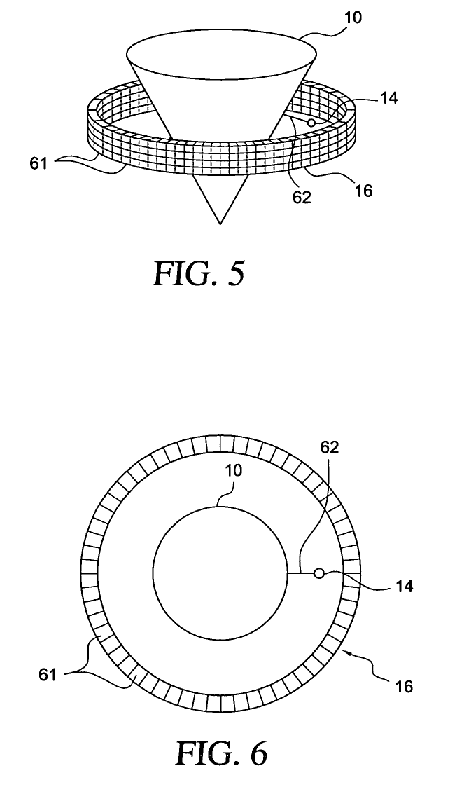

FIG. 5 shows the ring of detectors 16 made up of multiple rows of individual detectors 61 for laser scanning with the laser source 14. During a scan, the laser source 14 emits a pencil beam 62 of laser toward the breast 10. Upon hitting the breast, photons of the laser beam scatter in all directions. Some are absorbed inside the breast and some survive and exit the breast. Some of the surviving photons are detected by the detectors 61 of the ring, giving rise to the so called “optical data” used for image reconstruction.

The detectors 61 may be provided with optical filters to allow detection of photons only within a selected predefined narrow range of wave lengths. Optical filters are commonly used to detect fluorescent emission from the far-brighter excitation light of the laser source. These filters are usually interference filters, composed of many layers of optical material deposited on glass. The filters may be either bandpass or longpass filters and are disposed close to the optical detectors 61.

FIG. 6 is a schematic top view of the of FIG. 5 that shows the relative positions of the detectors 61 within the top row of detectors of the ring 16, as well as the relative positions of the detector ring 16 and the laser source 14 with respect to the position of the breast 10. Note that the laser source 14 is disposed within the detector ring 16. The ring of detectors 16 moves up and down vertically, while the laser source 14 rotates around the breast 10 to provide a helical path during scanning.

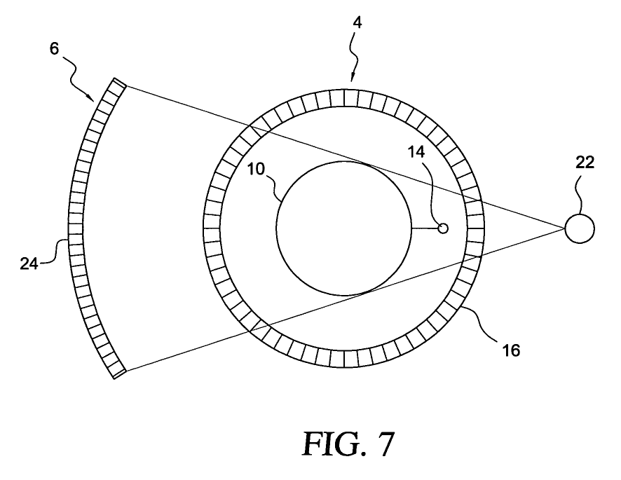

FIG. 7 provides a top view of the x-ray CT scanner 6 and laser CT scanner 4, showing the relative positions of the x-ray source 22, detector arc 24, laser source 14, detector ring 16 with respect to the position of the breast 10. Note that the laser detector ring 16 and the laser source 14 are disposed between the X-ray source 22 and the arc of detectors 24.

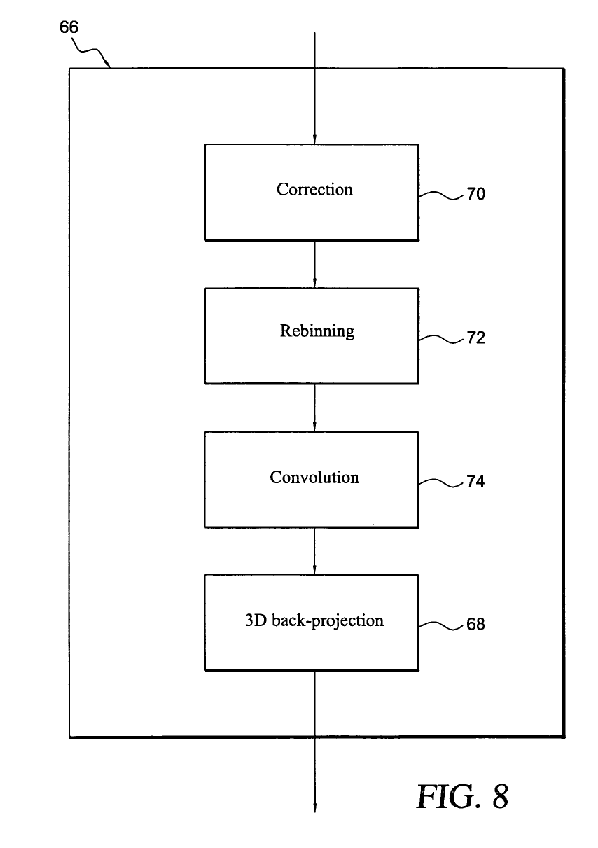

During a scan using x-ray CT scanner 6, the x-ray data acquisition system 36 collects a set of data representing information pertaining to the x-ray attenuation through the breast. In order to render this information easily readable, this data set is submitted to an image reconstruction process 66 shown in FIG. 8. The image reconstruction is done for the whole scanned volume of the breast. The scanned volume is embedded in a parallelepiped, which is subdivided into small volume elements known as voxels. The value of the reconstructed images at each voxel is the result of a 3D back-projection 88, as disclosed in Feldkamp et al., J. Opt. Soc. Am. A1, 612-619, 1984, incorporated herein by reference, or summation of data values along rays going through the 3D location of the voxel and the x-ray source locations when the source encircles the voxel in a period of π, along the helix. The data values contributing in the image reconstruction at each voxel are derived from the data collected by the X-ray data acquisition system 36. In order to ensure proper values of reconstructed images, the following data processing steps are applied to the collected data set before the 3D back-projection 68 takes place.
a) Data correction process 70 to correct for errors and inconsistencies introduced by various physical components utilized for the data acquisition. The source of errors and inconsistencies may include the mA variations of the high voltage generator to the x-ray source, the beam walk caused by non stable focal spot, non-linear and non-uniform response of detectors, non-perfect geometric locations to the x-ray sources or detectors.
b) Rebinning process 72 to derive from the above corrected data set another data set according to a different geometry underlying a fictive theoretical data collection. The choice of the new geometry is simply to facilitate a correct but simple 3D back-projection process 68. Parallel beam geometry is a preferred geometry for the present embodiment of the invention.
c) Convolution process 74 to ensure a sharp or less blurred resultant back-projected image. The convolution kernel may be a 2D or 1D filter derived from the ramp filter, as disclosed in Ramachandran et al., Proc. Nat. Acad. Sci. U.S., 68, 2236-2240, 1971, incorporated herein by reference. A 1D ramp filter is a preferred filter for the present invention.

The process 66 provides a preferred sequence of data processing starting from the raw data collected by the x-ray data acquisition system 36 to reconstructed images feeding to the visualization system 34.

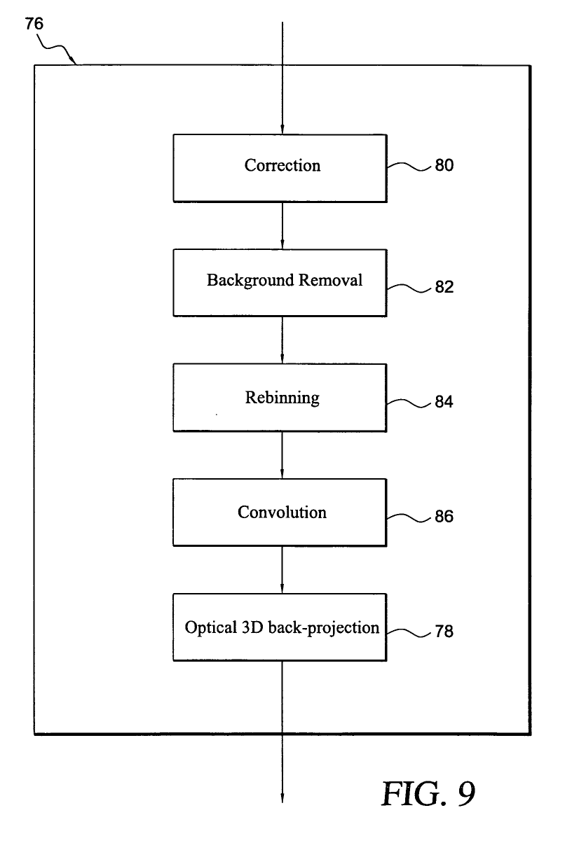

During a scan using the laser CT scanner 4, the laser data acquisition system 30 collects an optical data set representing information resulting from the scatter and absorption phenomena of the photons from the laser source 14 as they travel through the breast 10. In order to render this information easily readable, the optical data set is submitted to an image reconstruction process 76 shown in FIG. 9. Similar to the above reconstruction process 66 described for the x-ray system, the image reconstruction is done for whole scanned volume of the breast. The scanned volume is embedded in a parallelepiped, which is subdivided into small volume elements known as voxels. The value of the reconstructed images at each voxel results from an optical 3D back-projection 78 or simply a summation of data values along maximum probable photon paths, as disclosed in S. Feng et al., SPIE, 1888, 78-89, 1993, incorporated herein by reference, going through the 3D location of the voxel and the laser source locations as the source encircles the voxel in a period of 2π, along the helix. The data values contributing in the image reconstruction at each voxel are derived from the data collected by the laser data acquisition system 30. In order to ensure proper values of reconstructed images, the following data processing steps are applied to the collected data set before the 3D optical back-projection 78 takes place.
a) Data correction process 80 to correct for errors and inconsistencies introduced by various physical components utilized for the data acquisition. The source of errors and inconsistencies may include the power variations of the laser source, non-linear and non-uniform response of detectors, non-perfect geometric locations to the laser sources or detectors.
b) Background removal 82 to reveal the signal caused by inclusions inside the breast from the corrected data set. This background removal makes use of the data representing the boundary of the breast in order to estimate optical signal that would have had been detected if the laser source would have shined on a homogeneous media of the same dimensions as that of the breast. Background removal is a procedure of taking the log of the ratio between the optical measurements derived from the measurements from the scanner during the scan of the breast, and an estimation of uniform background derived from an estimation of solution of the homogeneous diffusion equation satisfying the boundary condition derived from the boundary of the breast, which was recorded during the scan.
c) Rebinning process 84 to derive from the above estimated perturbation signal, another data set according to a different geometry underlying a fictive theoretical data collection. The choice of the new geometry is simply to facilitate a correct but simple 3D optical back-projection process 78. Parallel beam geometry is a preferred geometry for the present invention.
d) Convolution process 86 to ensure a sharp or less blurred resultant back-projected image. The convolution kernel may be a 2D or 1D filter derived from the ramp filter, as disclosed in Ramachandran et al., Proc. Nat. Acad. Sci. U.S., 68, 2236-2240, 1971. A 1D ramp filter is a preferred filter in the present embodiment of the invention.

The process 76 provides a preferred sequence of data processing starting from the raw data collected by the laser data acquisition system 30 to reconstructed images feeding to the visualization system 34.

A scan generates a geometry, called scan geometry, described by the locations of the source (x-ray or laser), and the locations of all the detectors of the scanner when the signals are recorded and collected. The scan geometry is to indicate that data are known along the rays joining the source and detectors at the time that the data are recorded. For each instance of data collection—the rays along which data are recorded—are within a cone or a fan with the source position being its vertex. From this point of view the scan geometry consists of a set of fans or limited cone beams.

On the other hand, image reconstruction is done via a back-projection process, more precisely, the value of the reconstructed image at a voxel is the back-projection of convolved data at that voxel. As disclosed in co-pending application Ser. No. 11/494,534, filed Jul. 29, 2006, herein incorporated by reference, for the back-projection, it is advantageous to assume that data are known in a “curly wedge beam” geometry, which is different than the scan geometry. For this reason, it is required to synthesize data in the curly wedge beam geometry from data in the scan geometry before the convolution takes place. This process is known as the rebinning process.

The rebinning is done on a ray by ray basis. For a given ray in the wedge beam geometry, we look for 4 closest rays in the scan geometry. The data along that particular given ray is estimated by computing an interpolation of the collected data along these 4 closest rays. The coefficients of the interpolation is inferred from the relative location of the given ray with respect to its 4 closest rays, similar to what was disclosed in the co-pending application Ser. No. 11/494,534.

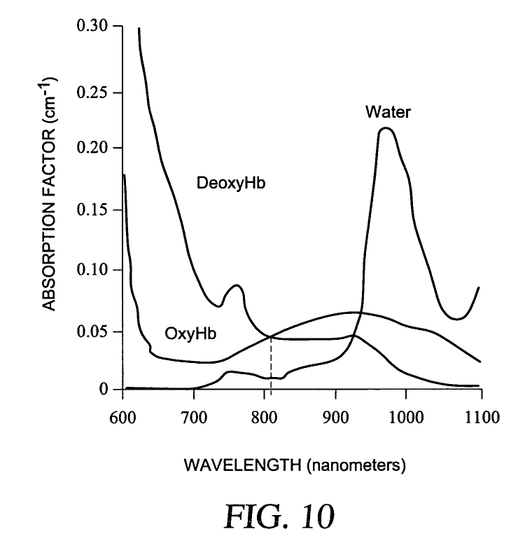

The scatter and absorption of photons during their travel through various tissues of the breast depend on the tissues and the wave length of the laser. It is advantageous for the user to be able to select the wave length of the laser in order to emphasize or deemphasize the structure he wants to view. A selection of a proper wave length may be based on the absorption factor curve as a function of materials and wave length. FIG. 10 shows an example of such curves (see V. Tuchin, Tissue Optics, SPIE Press, vol. TT38, 156-157, 2000). Based on these curves, the wave length in a neighborhood of 805 nm, which corresponds to an oxy-deoxyhemoglobin isosbestic point, is selected if both oxyhemoglobin and deoxyhemoglobin concentration areas are of the same informational value to the user. On the other hand, if the deoxyhemoglobin area is more important to view than the oxyhemoglobin area, then a wave length in a neighborhood of 760 nm would be selected.

The laser wavelength illuminating the patient may be selected electronically or mechanically, as is well known in the art. The outputs of multiple lasers could be optically combined, either via a fiber-optic combiner or via a series of dichroic mirrors, both techniques being well known in the optics industry. Then the lasers would be pulsed on sequentially via their respective controllers, giving a time-sequenced wavelength selection.

Alternatively, the lasers could be mechanically selected, via either a fiber-optic switch or via a galvanometer-controlled moving mirror, both being well known in the optics field.

While this invention has been described as having preferred design, it is understood that it is capable of further modification, uses and/or adaptations following in general the principle of the invention and including such departures from the present disclosure as come within known or customary practice in the art to which the invention pertains, and as may be applied to the essential features set forth, and fall within the scope of the invention or the limits of the appended claims.

We claim:

1. An apparatus for breast scanning to obtain functional and anatomical images of the breast comprising:
a) a patient support for a patient to rest in a prone position, said support having an opening with one of her breasts vertically pendent through said opening for scanning;
b) a laser CT scanner disposed below said support for generating first set of data for reconstruction of functional images of the breast;
c) an X-ray CT scanner disposed below said support for generating a second set of data for reconstruction of anatomical images of the breast; and
d) a display to visualize at least one of said functional and anatomical images.

2. An apparatus according to claim 1, wherein said patient support is shielded to limit exposure of the patient to X-ray radiation from said X-ray CT scanner.

3. An apparatus according to claim 1, wherein:
a) said laser CT scanner includes a laser source generating a beam directed toward the breast; and
b) a ring of detectors disposed around the breast to detect light after passing through the breast.

4. An apparatus as in claim 3, wherein said ring of detectors and said laser source are rotatable around said opening and movable vertically relative to said support and the breast.

5. An apparatus as in claim 4, wherein:
a) said laser source is mounted to a rotor of a bearing;
b) said ring of detectors is mounted to a stator of said bearing; and
c) vertical actuators connected to said stator for vertically moving said ring of detectors and said laser source.

6. An apparatus as in claim 1, wherein the wavelength of said laser source is selectable.

7. An apparatus as in claim 3, wherein said detectors each includes an optical filter to allow photons within a selected range of wavelengths to be detected.

8. An apparatus according to claim 1, wherein:
a) said X-ray CT scanner includes an X-ray source generating a limited cone X-ray beam directed toward the breast; and
b) X-ray detectors for detecting the X-ray beam after passing through the breast.

9. An apparatus as in claim 8, wherein:
a) said X-ray detectors and said X-ray source are rotatable around said opening and movable vertically relative to said support and the breast.

10. An apparatus as in claim 9, wherein:
a) said X-ray detectors and said X-ray source are mounted to a rotor of a bearing;
b) said detectors and said X-ray source are mounted to a stator of said bearing; and
c) vertical actuators connected to said stator for vertically moving said detectors and said X-ray source.

11. An apparatus as in claim 1, wherein said functional and anatomical images are fused.

12. An apparatus as in claim 1, wherein said X-ray source provides a limited cone radiation beam.

13. An apparatus as in claim 3, wherein:
a) said X-ray CT scanner includes an X-ray source generating a limited cone X-ray beam directed toward the breast;
b) X-ray detectors for detecting the X-ray beam after passing through the breast; and
c) said ring of detectors are disposed between said X-ray source and said X-ray detectors.

14. A method for acquiring data for reconstruction of images pertaining to functional and anatomical structures of a breast, comprising:
a) positioning a patient in a prone position on a support having an opening through which a breast of the patient is pendant;
b) scanning the breast with a laser CT scanner to obtain data of the breast for functional image reconstruction of the breast; and
c) while the patient is still prone on the support, scanning the breast with an X-ray CT scanner to obtain data of the breast for anatomical image reconstruction of the breast.

15. A method for breast scanning for obtaining functional and anatomical images of the breast, comprising:
a) positioning a patient in a prone position on a support having an opening through which a breast of the patient is pendant;
b) scanning the breast with a laser CT scanner to obtain optical data of the breast for functional image reconstruction of the breast;
c) while the patient is still prone on the support, scanning the breast with an X-ray CT scanner to obtain data for anatomical image reconstruction of the breast; and
d) reconstructing functional and anatomical images of the breast from the laser and X-ray scanner data.

16. A method as in claim 15, wherein the functional and anatomical images are fused.

17. A method as in claim 15, wherein the functional and anatomical images are viewed separately.

18. A method as in claim 15, wherein said scanning with the laser CT scanner and said scanning with the X-ray CT scanner are performed independently of each other.

19. A method as in claim 15, wherein said scanning with the laser CT scanner and said scanning with the X-ray CT scanner are performed concurrently of each other.

20. A method as in claim 15, wherein said scanning with the laser CT scanner and said scanning with the X-ray CT scanner are each performed in a helical path around the breast.

21. A method as in claim 15, wherein said scanning with the X-ray CT scanner is performed with a limited cone beam of X-ray radiation.

22. A method as in claim 15, wherein selecting a wavelength of the laser source to emphasize or deemphasize structure of the breast under examination.

23. A method as in claim 15, wherein said reconstructing functional images of the breast includes background removal by taking the log of a ratio between optical measurements derived from the measurements from the laser CT scanner during said scanning of the breast, and an estimation of uniform background derived from an estimation of solution of a homogeneous diffusion equation satisfying a boundary condition derived from the boundary of the breast, which was recorded during said scanning.

24. A method as in claim 15, wherein said reconstructing functional images of the breast includes optical back-projection along maximum probability photon paths.
